(19) United States
(12) Patent Application Publication
(10) Pub. No.: US 2026/0228845 A1
(43) Pub. Date: Aug. 6, 2026

(54) OACRA (Offender-Oriented Compliance and Rehabilitation App)

(71) Applicant: Abdelah Lomri, Davenport, FL (US)

(72) Inventor: Abdelah Lomri, Davenport, FL (US)

(21) Appl. No.: 19/183,865

(22) Filed: Apr. 20, 2025

Related U.S. Application Data
(60) Provisional application No. 63/755,153, filed on Feb. 6, 2025.

(51) Int. Cl. G06Q 50/26; G06Q 10/1093; G06Q 30/0207; G16H 20/70; H04W 12/63

(52) U.S. Cl. CPC G06Q 50/26; G06Q 10/1093; G06Q 10/1097; G06Q 30/0224; G16H 20/70; H04W 12/63

(57) ABSTRACT

The present invention relates to a digital system for offender compliance management and rehabilitation. OACRA is a mobile and web-based platform that enables individuals under legal supervision to independently manage court-ordered conditions using an AI-powered calendar, gamified progress tracking, and self-service tools. The system generates dynamic schedules, rewards compliance behaviors, enables voluntary GPS logging, and automates early termination eligibility reviews, effectively serving as a digital self-checkout model for legal supervision.

This application claims the benefit of U.S. Provisional Ser. No. 63/755,153 , filed Feb. 6, 2025, the contents of which are incorporated herein by reference.

### FIELD

The invention pertains to digital compliance tools, specifically to an offender-oriented automated system designed for use in the fields of criminal justice, probation management, and rehabilitation support.

### BACKGROUND

Millions of individuals on probation or parole struggle to manage complex supervision requirements such as court appearances, treatment participation, community service, and restitution. Current systems rely heavily on manual officer oversight and lack offender-facing tools for education, scheduling, and progress tracking.

Missed deadlines due to confusion or logistical challenges often lead to violations and re-incarceration. There is no known offender-facing system designed to automate and incentivize compliance while preserving personal privacy.

### SUMMARY

OACRA functions as a self-directed compliance management system, automating the scheduling, tracking, and reporting of court-ordered obligations. It enables the user to upload supervision terms, which are processed by an AI engine to generate a personalized schedule with alarm-enabled reminders. Through a dynamic calendar interface, users receive progress updates and can modify entries based on real-world changes.

The system includes a gamified incentive engine that awards points and badges for completed tasks, compliance streaks, and milestone achievements. Users may earn eligibility for early termination upon fulfilling supervision requirements. Voluntary GPS check-ins allow users to demonstrate location-based compliance without real-time tracking.

A resource referral module provides access to employment, housing, transportation, and court-approved classes by matching ZIP code with AI-analyzed conditions. Support features include a 24/7 chatbot and live counselor appointment scheduling.

OACRA mirrors the self-checkout model by enabling lower-risk individuals to independently navigate their supervision through a structured, guided platform, reducing the workload on supervising agencies and improving reentry outcomes.

### BRIEF DESCRIPTION OF THE DRAWINGS

FIG. 1 System architecture of the oacra platform.

FIG. 2 Incentivized self-checkout user flow from input to early termination eligibility.

FIG. 3 Government cost efficiency comparison between OACRA and traditional supervision.

FIG. 4 AI referral engine for court-ordered classes by ZIP code.

FIG. 5 Badge and point-based rewards dashboard interface for compliance tracking.

FIG. 6 Self-reported GPS compliance input and summary interface.

### DETAILED DESCRIPTION

At the core of OACRA's innovation is the concept of incentivized compliance shift away from punitive supervision models toward a proactive, motivational framework. Instead of relying solely on enforcement and threat of violation, OACRA encourages successful outcomes through positive reinforcement. The platform's gamified structure awards users with points, badges, and progression-based milestones for completing their court-ordered obligations on time or ahead of schedule. These rewards serve not only as visual markers of progress but also as psychological motivators, tapping into goal-driven behavior and a sense of autonomy.

The justice-tech landscape currently lacks a centralized platform that allows justice-involved individuals to navigate their court-ordered conditions and rehabilitation resources with autonomy and clarity. The existing ecosystem is fragmented: community service providers, treatment centers, housing programs, employment services, and court-approved courses are dispersed across independent systems and often lack a unified point of access. This fragmentation contributes to missed deadlines, confusion, and higher rates of non-compliance.

OACRA innovates by consolidating all critical compliance and rehabilitation services into a single application that is accessible by users based on their location. Through the referral engine, users can access relevant services filtered by state, county, city, and ZIP code. These services may include—but are not limited to—court-approved community service opportunities, defensive driving school enrollment, anger management classes, transitional housing programs, reentry-friendly employers, and transportation assistance. The user can explore, filter, and schedule appointments with providers directly from the platform.

The system ensures that each user only sees services aligned with their unique supervision terms and geographic constraints, minimizing risk of unintentional non-compliance. The AI categorizes and recommends services using embedded condition tags (e.g., “anger management,” “community service,” “employment”) linked to local resources. This structured integration is not only novel but also essential in reducing the administrative burden on probation officers and agencies while promoting successful rehabilitation outcomes.

The following describes the technical operation and interaction of each core module of the invention:
1. User Interface and Dashboard Module: The user interface is built with accessibility and clarity in mind. Upon logging in, the user is presented with a dynamic dashboard that displays active supervision tasks, pending deadlines, percentage of compliance progress, and current badge level. Visual indicators such as progress bars, alerts, and color-coding help users quickly identify which tasks are overdue or upcoming. The dashboard serves as a single-point hub for navigating the platform.
2. AI Calendar and Task Engine: Once the user inputs or uploads their supervision terms (e.g., a PDF or text field entry), the AI engine extracts relevant conditions using embedded rules and parsing logic. These conditions are converted into calendar tasks, with automatic frequency assignment (e.g., weekly check-in, monthly payment). Tasks are categorized (e.g., community service, financial, treatment) and color-coded. The calendar allows manual adjustments while preserving compliance logic.
3. Gamification Engine: Every supervised activity completed on time earns the user compliance points. Reaching specific thresholds unlocks tiered badges—Bronze, Silver, Gold—displayed on the dashboard. Missed tasks or late entries result in point deductions. For example, missing a drug test might deduct 15 points, whereas timely completion of 10 consecutive tasks could earn a bonus. This structure promotes consistent engagement and rewards positive behavior.
4. Early Termination Monitoring System: The AI continually evaluates the user's progress against standard and special conditions required for early termination eligibility. Once conditions are met (e.g., financial obligations paid, classes completed), the user receives a notification with a readiness report. The platform may auto-generate a request template or printable summary to submit to the supervising officer or court.
5. Voluntary GPS Check-in Tool: To ensure privacy, OACRA does not use passive location tracking. Instead, users may manually check in from approved locations such as work, class, or service providers. The system captures the time, location name, and task category. This feature adds an optional layer of accountability and is especially useful for verifying participation in unsupervised settings.
6. Compliance Report Generator: All actions taken by the user are logged in a structured timeline, which can be exported as a PDF or Excel file. Reports include completed hours, missed events, payments made, treatment logs, and location check-ins. These are formatted to align with standard probation report templates used by courts or supervision officers.
7. Referral Engine and Resource Directory: Upon account setup, the user is prompted to enter their ZIP code and supervision type. The engine queries the database of pre-verified providers and returns location-specific services such as housing, employment, substance abuse programs, anger management classes, and transportation support. Each listing includes eligibility tags, contact info, distance from user, and appointment options.
8. Support and Escalation Module: The platform includes an always-on chatbot to answer FAQs about supervision terms, deadlines, and resource access. If the user displays high-risk patterns (e.g., repeated missed tasks), the system triggers a soft escalation, prompting the user to schedule a session with a human counselor. The goal is to intervene supportively before a violation occurs.

### Example Use Case

John Doe begins probation in Orange County, FL. Upon onboarding, he uploads a PDF of his probation order, which includes conditions such as 50 hours of community service, weekly drug testing, anger management classes, and restitution.

The AI extracts and schedules all tasks in his calendar. John receives weekly reminders, logs his community service hours, and uploads receipts for payments. He checks in using GPS at the class location.

Each completed action awards points. After 3 months of consistent compliance, John earns a Silver badge and unlocks a goal tracker. The AI notifies him when he qualifies for early termination and generates a downloadable report for his probation officer.

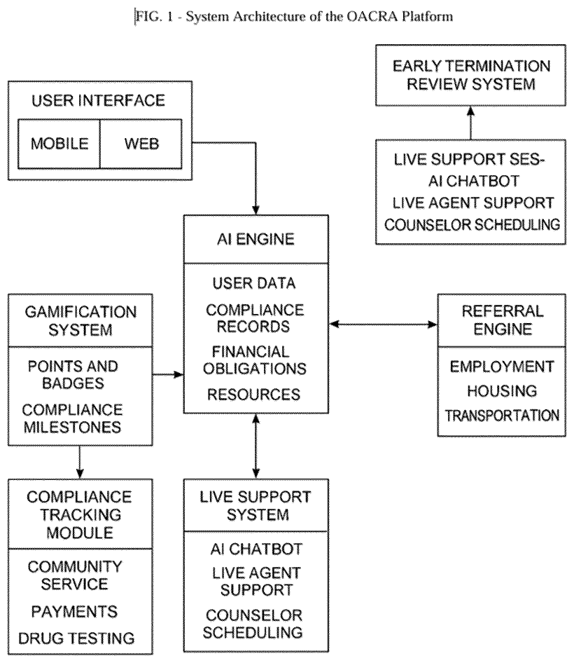

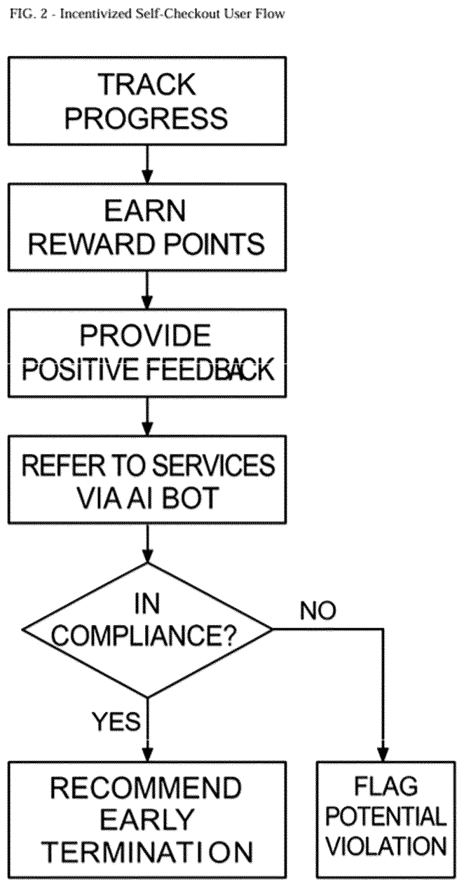

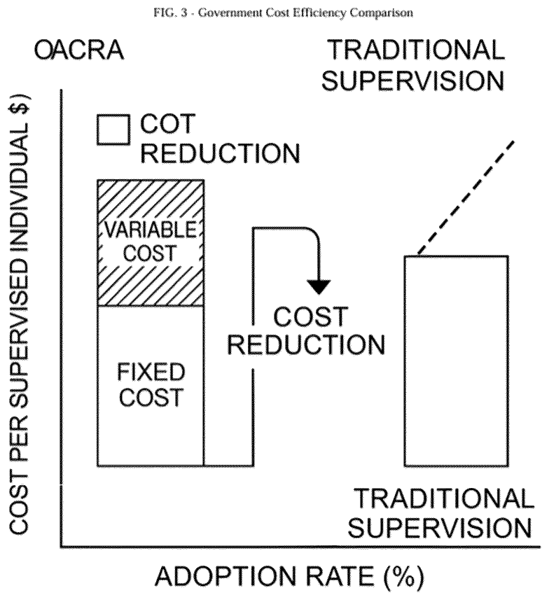

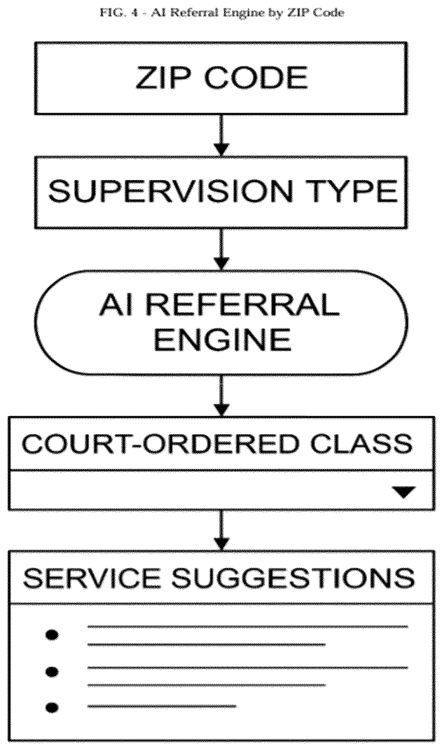

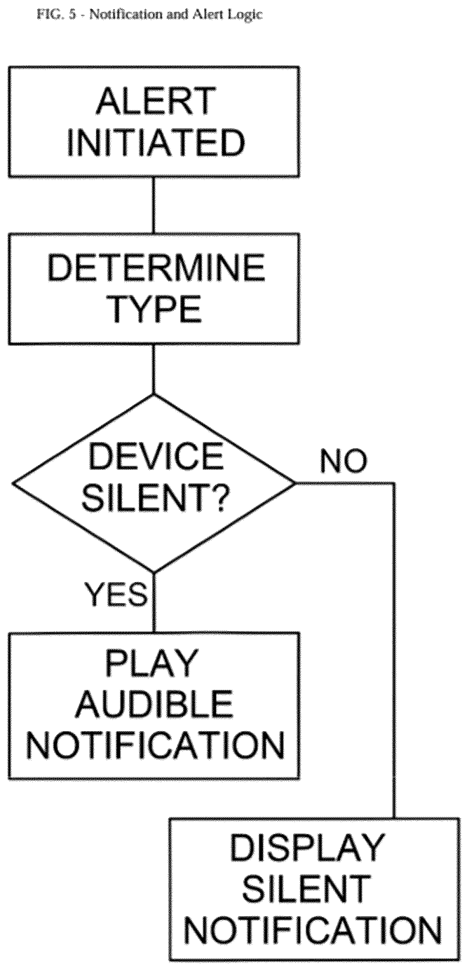

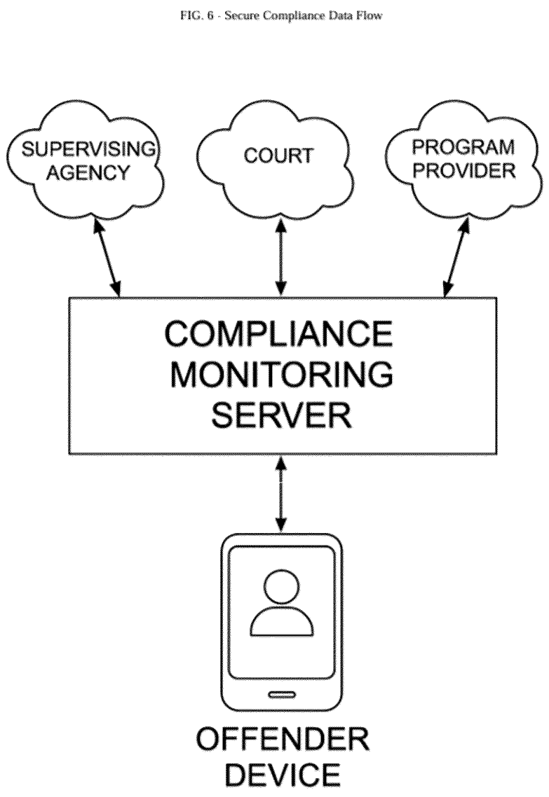

APPENDIX: Figures Include printed versions of each labeled figure: FIG. 1-6 in black and white line drawing format.

1. A system for managing court-ordered supervision and rehabilitation, comprising:
(a) a user interface configured to receive input of supervision conditions;
(b) a calendar module, integrated with an artificial intelligence engine, for auto-generating task schedules and appointment reminders based on the received supervision conditions;
(c) a gamification module configured to assign incentive-based rewards including points and badges for compliance milestones;
(d) a voluntary GPS check-in interface excluding continuous location tracking;
(e) a progress tracking engine for monitoring task completion, including community service hours, financial obligations, and treatment participation;
(f) a downloadable reporting module for compliance summaries;
(g) a referral module for location-based access to employment, housing, and support services;
(h) a support module comprising a chatbot and a live counselor appointment system; and
(i) a decision engine configured to evaluate early termination eligibility based on completed supervision tasks and user progress.

2. The system of claim 1, wherein the gamification module further comprises functionality to deduct points for non-compliance events while providing motivational messages encouraging rehabilitation.

3. The system of claim 1, wherein the referral module utilizes geographic data and supervision condition type to identify and recommend court-approved service providers.

4. The system of claim 1, wherein the downloadable reporting module generates a time-stamped activity log including completed tasks, upcoming deadlines, financial transactions, and earned rewards.

5. The system of claim 1, wherein the support module enables real-time communication with human counselors for personalized assistance in interpreting supervision conditions and accessing community resources.

6. The system of claim 1, wherein the artificial intelligence engine dynamically recalibrates the user's schedule based on manual edits and compliance progress.

7. The system of claim 1, wherein the user interface presents a dashboard displaying compliance percentage, progress bar, alerts for missed deadlines, and visual indicators of earned incentives.

8. The system of claim 1, wherein the voluntary GPS module stores location logs securely and allows users to associate check-ins with compliance tasks for validation without triggering constant monitoring.

9. The system of claim 1, wherein the gamification module assigns tiered badge levels based on accumulated point thresholds.

10. The system of claim 1, wherein the reporting module compiles a compliance summary exportable as a court-formatted PDF.

11. The system of claim 1, wherein the AI referral engine filters service suggestions by supervision condition keyword tags.

12. The system of claim 1, wherein the calendar module includes recurring task logic and tracks missed events.

13. The system of claim 1, wherein the support module includes automated escalation to live counselor intervention after flagged high-risk patterns.

14. The system of claim 1, wherein the early termination engine is configured to apply jurisdictional thresholds dynamically based on uploaded supervision terms.

15. The system of claim 1, wherein the compliance tracking engine logs progress toward multiple concurrent conditions and assigns weighted priority scores to each.
